SEQUENCE LISTING (2 sequences)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - DNA, 3428 bp, Homo sapiens
tggagagtaatgttacagagcggagagagtgaggaggctgcgtctggctcccgctctcacagccattgcagtacattgagctccatagagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttccattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagaggttttaaatactagttaaatggctttcacttaagaacttaagattttgttacatatttttaaatcttgtttctaataatacctcttagcagtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacgtcatgacaactaccatttttttaagatgttgagaatgggaacagtttttttagggtttattcttgaccacagatcttaagaaaatggacaaaacccctcttcaatctgaagattagtatggtttggtgttctaacagtatcccctagaagttggatgtctaaaactcaagtaaatggaagtgggaggcaatttagataagtgtaaagccttgtaactgaagatgattttttttagaaagtgtatagaaactattttaatgccaagatagttacagtgctgtggggtttaaagactttgttgacatcaagaaaagactaaatctataattaattgggccaacttttaaaatgaagatgctttttaaaactaatgaactaagatgtataaatcttagtttttttgtattttaaagataggcatatggcatattgattaacgagtcaaatttcctaactttgctgtgcaaaggttgagagctattgctgattagttaccacagttctgatgatcgtcccatcacagtgttgttaatgtttgctgtatttattaattttcttaaagtgaaatctgaaaaatgaaatttgtgtgtcctgtgtacccgaggggtaatgattaaatgataaagataagaaaa

(19) United States
(12) Patent Application Publication
(10) Pub. No.: US 2011/0123483 A1
(43) Pub. Date: May 26, 2011

(54) HMGB1 FOR CANCER TREATMENT

(75) Inventors: Wilfried ROTH, Schwetzingen (DE); Georg GDYNIA, Heidelberg (DE)

(73) Assignee: DEUTSCHES KREBSFORSCHUNGSZENTRUM, Heidelberg (DE)

(21) Appl. No.: 12/943,525

(22) Filed: Nov. 10, 2010

Related U.S. Application Data
(60) Provisional application No. 61/263,750, filed on Nov. 23, 2009.

(51) Int. Cl. A61K 38/19; A61K 31/7088; A61K 48/00; A61K 35/12; A61P 35/00

(52) U.S. Cl. 424/85.1; 514/44 R; 424/93.7

(57) ABSTRACT

The current invention relates to a method for treating a subject suffering from cancer comprising administering to said subject a therapeutically effective dose of High Mobility Group B1 (HMGB1) protein or of a polynucleotide comprising a coding region for an HMGB1 gene in an expressible form. Moreover, it relates to the combination of said method with other cancer treatment regimens.

The current invention relates to a method for treating a subject suffering from cancer comprising administering to said subject a therapeutically effective dose of High Mobility Group B1 (HMGB1) protein or of a polynucleotide comprising a coding region for an HMGB1 gene in an expressible form. Moreover, it relates to the combination of said method with other cancer treatment regimens.

The High Mobility Group B1 (HMGB1) protein belongs to the High Mobility Group (HMG) family of nuclear proteins, which was named due to the unusual high mobility of its members in SDS-polyacrylamide gelelectrophoresis (SDS-PAGE). These proteins are, second to histones, among the most abundant proteins associated with chromatin and they play an architectural role in the nucleus of the eikaryotic cell in that they bend, distort or otherwise modify the conformation of DNA, thereby also modifying the binding of transcription factors to DNA. HMG proteins have been implicated in the genesis of various disorders, like several kinds of benign tumors and autoimmune diseases.

For the HMGB1 proteins, several structural motifs have been described: two DNA-binding domains (box A and box B), two nuclear localization sequences, and a C-terminal acidic domain. The HMGB1 proteins can be extensively posttranslationally modified by acetylation, methylation, ADP-ribosylation, phosphorylation or glycosylation. Acetylation of the nuclear localization sites is the signal that causes the HMGB1 protein to be actively secreted from activated cells of the immune system. Besides active secretion, HMGB1 is also released passively from necrotic cells.

Extracellular HMGB1 is a potent cytokine and a strong activating factor for macrophages and other cells of the immune system, leading to an extensive inflammatory reaction. For this reason, HMGB1 has been implicated in autoimmune diseases such as systemic lupus erythematosus and rheumatoid arthritis. However, high amounts of HMGB1 in blood have also been found to indicate serious or life-threatening inflammatory conditions like sepsis. To antagonize such HMGB1 related pathologies, inhibitors of HMGB1 function, like inhibitory antibodies or fragments thereof, variants of HMGB1 comprising mutations in box A, or polymer conjugates of the box A domain have been described (U.S. Pat. No. 6,468,533, WO 02/074337, US 2003/0144201, WO 2006024547, and WO 2008031612).

Cancer treatment, besides surgical removal of tumor tissue, essentially relies on the application of medicaments and/or treatments that exert a deleterious function on actively dividing cells. By its nature, such treatment will also harm non-tumor cells and tissues undergoing cell division in the human body, leading to most of the well-known and dreaded side effects of chemo- and radiotherapy, like nausea, digestive distortions, fatigue, hair loss, and more. It is, thus, desirable to have new therapeutic agents at hand that are effective via hitherto unknown routes of action, thereby potentially allowing a dose reduction in chemo- and/or radiotherapy, alleviating side-effects. The provision of such agents using new routes of cancer cell killing could also potentially contribute to the removal of cancer stem cells, which can survive chemotherapy by falling into a resting state and which were recently found to be responsible for at least a fraction of all relapses and metastases.

Tumors have also been known to suppress immune reactions, especially at the site of the tumor itself, leading to immune evasion of tumors that would otherwise be attacked by the immune system. So there is also a need in the art to provide means and methods to activate the immune system in cancer patients, so as to increase the chance of the immune system removing tumor cells.

Thus, the present invention relates to a method for treating a subject suffering from cancer comprising administering to said subject a therapeutically effective dose of High Mobility Group B1 (HMGB1) protein.

As used herein, the term “method for treating” refers to performing actions that lead to amelioration of the cancers referred to herein or of the symptoms accompanied therewith to a significant extent. The combination of said actions is related to as “treatment”. Amelioration of a cancer, preferably, comprises a reduction in the number of cancer cells in a subject or a reduction in the number of cancer cells at a specific site in the body of a subject. Said treatment as used herein also includes an entire restoration of the health with respect to the cancers referred to herein. It is to be understood that treatment as used in accordance with the present invention may not be effective in all subjects to be treated. However, the term shall require that a statistically significant portion of subjects suffering from a cancer referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population.

Preferably, the method of treatment comprises administering HMGB1 protein as specified herein below. More preferably, the method further comprises administering least one chemotherapeutic agent and/or at least one biotherapeutic agent and/or at least one virotherapeutic agent. A “chemotherapeutic agent” as used herein is a chemical compound used in the chemotherapy of cancer, i.e. a chemical agent or drug that is destructive malignant cells and tissues. Chemotherapeutic agents can be comprised in various groups, like e.g. alkylating agents (e.g. cisplatin, carboplatin, oxaliplatin), anti-metabolic agents (e.g. thioguanine, Fludarabine, Pentostatin, Cladribine, 5-Fluoruracil, Gemcitabine), plant-derived agents (e.g. Vincristine, Vinblastine, etoposide, paclitaxel, Docetaxel), topoisomerase inhibitors (e.g. camptothecins like irinotecan and topotecan; amsacrine), or antineoplastics (e.g. bleomycin).

A “biotherapeutic agent” as used herein relates to a biological oligo- or polymeric molecule, comprising e.g. amino acids, nucleotides, lipids and/or saccharides, said molecule being destructive to malignant cells and tissues. Examples of biotherapeutic agents include inhibitory antibodies and fragments thereof, agonists and antagonists of intra- or extracellular receptors, ribozymes, siRNAs, peptide and nucleic acid aptamers or dominant negative derivatives of cellular effectors.

A “virotherapeutic agent” as used herein relates to a virus or to a biotherapeutic agent derived from a virus, said virus or molecule being destructive to malignant cells or tissues. Said virus may be a wildtype virus or a genetically modified virus. Examples of viruses that can be used as virotherapeutic agents are parvovirus H1, measles virus, or foamy virus; genetically modified viruses include retroviruses like HIV, adenovirus, herpesviruses, and the like. Genetically modified viruses may, preferably, comprise in addition a HMGB1 polynucleotide as specified herein below. Examples for virus-derived molecules are the herpesviral thymidine-kinase, the parvovirus NS1 cytotoxin, the apoptin protein derived from chicken aneamia virus, mengovirus- or poliovirus-derived RNA replicons, Semliki Forest virus LacZ particles.

Preferably, the method of the current invention further comprises surgical treatment and/or radiotherapy. “Surgical treatment” as meant herein relates to treatment of cancer by manual and instrumental means, e.g. to removal of tumor and/or metastasis tissue by operative methods. “Radiotherapy” relates to treatment of cancer by means of x-rays or of radioactive substances, e.g. to destroy tumor cells.

A “subject suffering from cancer” as used herein relates to a mammal, preferably a human, afflicted with a disease characterized by uncontrolled growth by a group of body cells (“cancer cells”). This uncontrolled growth may be accompanied by intrusion into and destruction of surrounding tissue and possibly spread of cancer cells to other locations in the body. Preferably, the cancer is an acute lymphoblastic leukemia, an acute myeloid leukemia, an adrenocortical carcinoma, an aids-related lymphoma, an anal cancer, an appendix cancer, an astrocytoma, an atypical teratoid, a basal cell carcinoma, a bile duct cancer, a bladder cancer, a brain stem glioma, a breast cancer, a burkitt lymphoma, a carcinoid tumor, a cerebellar astrocytoma, a cervical cancer, a chordoma, a chronic lymphocytic leukemia, a chronic myelogenous leukemia, a colorectal cancer, a craniopharyngioma, an endometrial cancer, an ependymoblastoma, an ependymoma, an esophageal cancer, an extracranial germ cell tumor, an extragonadal germ cell tumor, an extrahepatic bile duct cancer, a gallbladder cancer, a gastric cancer, a gastrointestinal stromal tumor, a gestational trophoblastic tumor, a hairy cell leukemia, a head and neck cancer, a hepatocellular cancer, a hodgkin lymphoma, a hypopharyngeal cancer, a hypothalamic and visual pathway glioma intraocular melanoma, a kaposi sarcoma, a laryngeal cancer, a medulloblastoma, a medulloepithelioma, a melanoma, a merkel cell carcinoma, a mesothelioma, a mouth cancer, a multiple endocrine neoplasia syndrome, a multiple myeloma, a mycosis fungoides, a nasal cavity and paranasal sinus cancer, a nasopharyngeal cancer, a neuroblastoma, a non-hodgkin lymphoma, a non-small cell lung cancer, an oral cancer, an oropharyngeal cancer, an osteosarcoma, an ovarian cancer, an ovarian epithelial cancer, an ovarian germ cell tumor, an ovarian low malignant potential tumor, a pancreatic cancer, a papillomatosis, a paranasal sinus and nasal cavity cancer, a parathyroid cancer, a penile cancer, a pharyngeal cancer, a pheochromocytoma, a pituitary tumor, a pleuropulmonary blastoma, a primary central nervous system lymphoma, a prostate cancer, a rectal cancer, a renal cell cancer, a retinoblastoma, a rhabdomyosarcoma, a salivary gland cancer, a sézary syndrome, a small cell lung cancer, a small intestine cancer, a soft tissue sarcoma, a squamous cell carcinoma, a squamous neck cancer, a testicular cancer, a throat cancer, a thymic carcinoma, a thymoma, a thyroid cancer, an urethral cancer, an uterine sarcoma, a vaginal cancer, a vulvar cancer, a waldenström macroglobulinemia, or a wilms tumor. More preferably, the cancer is a glioblastoma or a colon carcinoma.

It is to be understood that “suffering” as meant herein does not necessarily mean enduring pain, disability, or an other unpleasant state that may be of relevance in the context of cancer, but solely states the fact that a person is afflicted with cancer.

The term “administering” relates to applying a compound in a composition and via a route suitable to achieve treatment of the cancers related to herein. Preferably, suitable routes of administration are peroral, oral, topical, or parenteral (e.g. intramuscular, intrathecal, subcutaneous, transdermal, intrapulmonal, intravesical, intraperitoneal, intracardial, intravaginal, intravitreal) administration, more preferably a compound is administered intravenously or intratumorally. However, depending on the nature and mode of action of a compound, the pharmaceutical compositions may be administered by other routes as well. For example, the polynucleotide compounds of this invention may be administered in a gene therapy approach by using viral vectors or viruses or liposomes.

The compounds are, preferably, administered in conventional dosage forms prepared by combining the compounds with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Moreover, the compounds can be administered in combination with other therapeutic agents as specified herein below either in a common pharmaceutical composition or as separated pharmaceutical compositions wherein said separated pharmaceutical compositions may be provided in form of a kit of parts. Administration of separated pharmaceutical compositions a can be simultaneous or at different times in the treatment.

A “therapeutically effective dose” refers to an amount of the compounds of the present invention which prevents, ameliorates or treats the symptoms accompanying a cancer referred to in this specification. Therapeutic efficacy and toxicity of the compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depend upon many factors, including the patient's size, body surface area, age, the particular compound to be administered, sex, time and route of administration, general health, and other drugs being administered concurrently. Progress can be monitored by periodic assessment. A typical dose can be, for example, in the range of 0.1 to 1000 mg; however, doses below or above this exemplary range are envisioned, especially considering the aforementioned factors. Generally, the regimen as a regular administration of the compounds should be in the range of 1 μg to 10 mg units per day. If the regimen is a continuous infusion, it should also be in the range of 1 μg to 10 mg units per kilogram of body weight per minute, respectively. Progress can be monitored by periodic assessment. However, depending on the subject and the mode of administration, the quantity of substance administration may vary over a wide range to provide from about 0.01 mg per kg body mass to about 10 mg per kg body mass, preferably.

The compounds referred to herein are administered at least once in order to treat or ameliorate or prevent a disease or condition recited in this specification. However, said compounds may be administered more than one time, for example from one to four times daily up to a non-limited number of days.

As used herein, the term “High Mobility Group B1 protein” (HMGB1 protein) relates to the human HMGB1 protein (Genbank ACC No: NP_{—}002119.1 GI:4504425, SEQ ID NO: 1) or partial sequences thereof having the activity of inducing cell death. Suitable assays for measuring the activities mentioned before are described in the accompanying Examples or in (Kroemer, G., et al. Classification of cell death: recommendations of the Nomenclature Committee on Cell Death 2009. Cell Death Differ 16, 3-11 (2009) and Galluzzi, L., et al. Cell death modalities: classification and pathophysiological implications. Cell Death Differ 14, 1237-1243 (2007)). The HMGB1 protein may be purified from cells or tissues or it may be chemically synthesised or, preferably, can be recombinantly manufactured. The HMGB1 protein may comprise further amino acids which may serve as a tag for purification or detection. Moreover, the HMGB1 protein may be comprised by a fusion polypolypeptide.

In a preferred embodiment of the HMGB1 protein, the HMGB1 protein further comprises a detectable tag. The term “detectable tag” refers to a stretch of amino acids which are added to or introduced into the HMGB1 protein. Preferably, the tag shall be added C- or N-terminally to the HMGB1 protein. The said stretch of amino acids shall allow for detection of the HMGB1 protein by an antibody which specifically recognizes the tag or it shall allow for forming a functional conformation, such as a chelator or it shall allow for visualization by fluorescent tags. Preferred tags are the Myc-tag, FLAG-tag, 6-His-tag, HA-tag, GST-tag or GFP-tag. These tags are all well known in the art.

Preferably, the term “HMGB1 protein” includes variants of said amino acid sequence, said variants having an amino acid sequence being at least 70%, at least 80%, at least 90%, at least 95%, least 96%, at least 97%, at least 98% or at least 99% identical to the amino acid sequence of the human HMGB1 protein as specified above (SEQ ID NO: 1), however, the said variants retain the function of inducing cell death. The percent identity values are, preferably, calculated over the entire amino acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981)), which are part of the GCG software packet (Genetics Computer Group, 575 Science Drive, Madison, Wis., USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000.

Moreover, HMGB1 protein further encompasses variants of the aforementioned specific amino acid sequence which may represent orthologs, paralogs or other homologs of the human HMGB1 protein. The variants, preferably, comprise an amino acid sequence characterized in that the sequence can be derived from the aforementioned sequence of human HMGB1 protein described above by at least one amino acid substitution and/or addition and/or deletion.

The term HMGB1 protein also includes chemically modified polypeptides, e.g., polypeptides containing modified amino acids or polypeptides which are, e.g., biotinylated, or are coupled to fluorophores, such as fluorescin, or Cy 3, are conformationally restricted, e.g. by disulfide bridging or by stapling (Walensky 2004, Science 305(5689): 1466-1470), or are linked to cell penetration polypeptides or protein transduction domains (Snyder 2004, Pharm Res 21(3): 389-393). Such modifications may improve the biological properties of the polypeptides, e.g., cell penetration, binding, stability, or may be used as detection labels.

The variant or modified HMGB1 proteins, preferably, retain the biological activity of the HMGB1 protein. The conservation of activity can be tested by the one skilled in the art by testing if the variant is able to induce cell death in cells as specified in example (FIG. 1 B-D).

The definitions made above apply mutatis mutandis to the following:

In a further preferred embodiment, the current invention relates to a method for treating a subject suffering from cancer comprising administering to said subject a therapeutically effective dose of a polynucleotide comprising an expressible coding region for an HMGB1 gene.

The term “polynucleotide comprising a coding region for an HMGB1 gene” (HMGB1 polynucleotide) as used herein relates to a polynucleotide comprising a nucleic acid sequence of a human HMGB1 gene (Genbank ACC No: NM_{—}002128.4 GI:118918424, SEQ ID NO: 2) which encodes a polypeptide having the activity described herein above. It is to be understood that an HMGB1 polypeptide having an amino acid sequence as shown above may be also encoded due to the degenerated genetic code by other polynucleotides as well. Moreover, the HMGB1 polynucleotide of present invention further encompasses variants of the aforementioned specific polynucleotides. Said variants may represent orthologs, paralogs or other homologs of the HMGB1 polynucleotide of the present invention. The polynucleotide variants, preferably, comprise a nucleic acid sequence characterized in that the sequence can be derived from the aforementioned HMGB1 polynucleotide by at least one nucleotide substitution, addition and/or deletion, whereby the variant nucleic acid sequence shall still encode a polypeptide having the activity as specified above. Variants also encompass polynucleotides comprising a nucleic acid sequence which is capable of hybridizing to the aforementioned HMGB1 polynucleotide, preferably, under stringent hybridization conditions. These stringent conditions are known to the skilled worker and can be found in Current Protocols in Molecular Biology, John Wiley & Sons, N.Y. (1989), 6.3.1-6.3.6. A preferred example for stringent hybridization conditions are hybridization conditions in 6× sodium chloride/sodium citrate (═SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, HMGB1 polynucleotide variants are obtainable by PCR-based techniques such as mixed oligonucleotide primer-based amplification of DNA, i.e. using degenerated primers against conserved domains of the polypeptides of the present invention. Conserved domains of the polypeptide of the present invention may be identified by a sequence comparison of the nucleic acid sequence of the polynucleotide or the amino acid sequence of the polypeptide of the present invention with sequences of other HMGB1 proteins. As a template, DNA or cDNA from bacteria, fungi, plants or animals may be used. Further, variants include polynucleotides comprising nucleic acid sequences which are at least 70%, at least 75%, at least 80%, at least 85%, at least 90%, at least 95%, at least 98% or at least 99% identical to the nucleic acid sequence specified above. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequence of to human HMGB1 protein as specified above (SEQ ID NO: 2). The percent identity values are, preferably, calculated over the entire amino acid or nucleic acid sequence region. Programs available to the skilled worker for comparing different sequences have been described herein above.

A polynucleotide comprising a fragment of any of the aforementioned nucleic acid sequences is also encompassed as a HMGB1 polynucleotide of the present invention. The fragment shall encode a polypeptide which still has the activity as specified above. Accordingly, the polypeptide may comprise or consist of the domains of the polypeptide of the present invention conferring the said biological activity. A fragment as meant herein, preferably, comprises at least 50, at least 100, at least 250 or at least 500 consecutive nucleotides of any one of the aforementioned nucleic acid sequences or encodes an amino acid sequence comprising at least 20, at least 30, at least 50, at least 80, at least 100 or at least 150 consecutive amino acids of any one of the aforementioned amino acid sequences.

The HMGB1 polynucleotides of the present invention either essentially consist of the aforementioned nucleic acid sequences or comprise the aforementioned nucleic acid sequences. Thus, they may contain further nucleic acid sequences as well. Specifically, the HMGB1 polynucleotides of the present invention may encode fusion proteins wherein one partner of the fusion protein is a polypeptide being encoded by a nucleic acid sequence recited above. Such fusion proteins, preferably, may comprise as additional part polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and comprise FLAG-tags, 6-histidine-tags, MYC-tags and the like.

The HMGB1 polynucleotide of the present invention shall be provided, preferably, either as an isolated polynucleotide (i.e. isolated from its natural context) or in genetically modified form. The HMGB1 polynucleotide, preferably, is DNA including cDNA or RNA. The term encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides.

As used herein, the term “expressible form” relates to the HMGB1 polynucleotide of the present invention being comprised in a polynucleotide in a form that allows a HMGB1 polypeptide to be expressed by a cell comprising said polynucleotide. Preferably, the HMGB1 polynucleotide is operatively linked to at least one expression control sequence allowing expression in prokaryotic or eukaryotic cells or isolated fractions thereof. Expression of said HMGB1 polynucleotide may comprise transcription of the polynucleotide, preferably into a translatable mRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide.

The term “vector” as used in this specification, preferably, encompasses phage, plasmid, viral or retroviral vectors as well artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells.

More preferably, the vector of the invention comprises the HMGB1 polynucleotide in an expressible form as specified herein above. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (InVitrogene) or pSPORT1 (GIBCO BRL). Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into the targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The term “host cell”, preferably, relates to a cell compatible with being administered to a subject. More preferably, said cell is immunologically compatible with the subject. Most preferably, the cell is a cell which was obtained from said subject. The host cell of the current invention, preferably, is a cell with a tendency to migrate into the vicinity of cancer cells, More preferably, the host cell is an immune cell, and most preferably is a cell of the immune system specifically recognizing a tumor specific antigen, like, e.g. a tumor antigen specific T-cell.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### FIGURE LEGENDS

FIG. 1: Cytotoxic effects of rhHMGB1 on human glioblastoma cells.

(A) U251MG glioma cells were treated with rhHMGB1 at concentrations as indicated (n=4; +/−SD). Cell numbers were determined by counting in a Neubauer chamber. (B) U251 MG glioma cells and non-neoplastic astrocytes were treated with rhHMGB1 for 72 h (n=3; +/−SD). Cellular survival was assessed by crystal violet staining. (C) Cotreatment of U251 MG glioma cells with 4 nM rhHMGB1 and TRAIL (24 h, n=3; +/−SD) or temozolomide (72 h, n=3; +/−SD). (D) Systemic (intraperitoneal) rhHMGB1 treatment of CD1 nude mice bearing U251MG xenograft tumors was started 3 weeks after inoculation of tumor cells (time point 0). Control mice were injected with saline. The tumor volumes were determined at regular intervals (n=4; +/−SD).

FIG. 2

Cytotoxic effects of rat recombinant HMGB1 (160 nM, 24 h; expressed in E. coli) on U251 MG glioma cells as assessed by crystal violet staining.

FIG. 3: Role of mitochondria, ROS generation, and JNK activation in rhHMGB1-induced cell death.

(A) ROS emission after treatment with rhHMGB1 was measured in U251 MG cells by DCF fluorescence and FACS analysis (p<0.05, n=3; +/−SD). (B) Survival of U251MG cells after 48 h treatment with rhHMGB1 and/or NAC (1 mM) as determined by crystal violet analysis. auto=autofluorescence control. (C) Western blot analysis of JNK1/2 phosphorylation in cells treated with rhHMGB1 (24 h). TNF-a served as a positive control for phosphorylation of JNK1/2. (D) Survival of U251 MG cells after 48 h treatment with rhHMGB1 in the presence or absence of the JNK inhibitor SP600125 (10 μM). (E) Effect of the ROS scavenger NAC on the phosphorylation of JNK1/2 in cells treated with rhHMGB1 (40 nM). (F) Survival of U251MG cells after 48 h treatment with rhHMGB1 and/or uridine (5 mg/ml). Pseudo rho zero cells were highly protected from rhHMGB1-induced cell death as assessed by crystal violet staining.

FIG. 4

Western blot analysis of JNK1/2 phosphorylation in U251 MG whole cell lysates (25 μg loaded) after treatment with UV light (10 min, 1.0 J/m2/s) or with rhHMGB1 (80 nM, 24 h). JNK activation is blocked by pre-treatment with the JNK inhibitor SP600125 (10 μM, 1 h).

FIG. 5

Survival of pseudo rho zero cells treated with rhHMGB1 (160 nM) for 24 h was assessed by crystal violet analysis. Treatment with the death ligand TRAIL (100 ng/ml for 24 h) served as a positive control for cytotoxicity (p<0.05, n=4).

FIG. 6

Subcellular fractionation and Western blot analysis from whole cell lysates of U251 MG cells (25 μg loaded). Release of cytochrome c into the cytosolic fraction (C) was detected after treatment of cells with the death ligand TRAIL (200 ng/ml, 3 h), but not after treatment with rhHMGB1 (left panel, 80 nM, 24 h), although rhHMGB1 at this concentration induces substantial cell death after 48 h. M=mitochondrial fraction. Similarly, cleavage of caspase 3 is observed after TRAIL treatment, but not after rhHMGB1 treatment (right panel).

FIG. 7

Expression levels of the proteins phospho-H2A.X and BNIP3 by Western blot analysis. Whole cell lysates of U251 MG cells (25 μg loaded) were generated after treatment with rhHMGB1 (24 h). Treatment with TNF-a (100 ng/ml, 1 h) served as a positive control.

FIG. 8: rhHMGB1 localizes to mitochondria and induces the formation of megamitochondria.

(A) Subcellular fractionation of U251MG cells treated with I-125-labelled HMGB1. The mitochondrial fraction showed a time-dependent increase in radioactivity. 1-125-labelled rhHMGB1 also accumulated in the nucleus (approximately 1.5 Mcpm in 4 h, data not shown). (B) Confocal immunofluorescence imaging of U251MG cells shows a colocalization of rhHMGB1 (40 nM for 24 h) with MitoTracker (50 nM for 30 min) accumulating in vital mitochondria.

FIG. 9

(A) The formation of megamitochondria was visualized by staining of COX IV in U251MG xenograft tissue (after a 6 week treatment with rhHMGB1). Arrows point to megamitochondria. Magnifications: 40×, upper panel; 140×, lower panel. (B) Macroscopic view of subcuatenous xenograft tumors (bar=1 cm).

FIG. 10: RhHMGBI-induced formation of giant mitochondria.

Electron microscopy of U251 MG cells after treatment with rhHMGBI (24 h). In control cells, the ultrastructure of mitochondria is maintained with numerous cristae and a dense matrix (left panels). In contrast, treatment with rhHMGBI results in the formation of massively enlarged mitochondria with pale matrix and a nearly complete lack of cristae (middle and right panels; asterisks: double membranes, C: rudimentary cristae; bar=1 um, bar in inset=200 nm).

FIG. 11: Effects of rhHMGB1 on mitochondrial DNA and proteins.

(A) Effect of rhHMGB1 on mitochondrial DNA: U251 MG cells were treated with rhHMGB1 (24 h) followed by isolation of mitochondrial DNA. In the dot blot analysis, the indicated amounts of DNA were loaded in a volume of 1 μl ddH2O. (B) The mitochondrial protein fractions (100 μg) from U251 MG cells treated with rhHMGB1 (80 nM, 24 h) or left untreated were visualized by 2-D gel electrophoresis and subsequent silver staining. Successful subcellular fractionation was confirmed by western blot (data not shown). (C) Mitochondrial protein fractions were subjected a 2-D gel electrophoresis as in (B). Then, the membrane was stained with anti-malondialdehyde antibody.

FIG. 12: Effect of rhHMGB1 on cancer stem cells

A) Effect of rhHMGB1 on the permeability of NCH241 glioblastoma stem cells to trypan blue. B) Comparison of cell morphology of NCH241 glioblastoma stem cells treated with the indicated concentrations of rhHMGB1. C) Propidiumiodide (PI) uptake in NCH421 K cells treated with 80 nM or 600 nM rhHMGB1 compared to the untreated control using FACS analysis.

### EXAMPLES

### Example 1

### Material and Methods

Cell culture

U251 glioblastoma cells and non-immortalized human astrocytic cells (ScienCell, USA) were maintained in DMEM (Life Technologies) containing 10% FCS, 4.5 g/L glucose, glutamine, 110 mg/L sodium pyruvate, and 1% penicillin-streptomycin (Life Technologies) and in astrocyte medium (ScienCell) containing 2% FBS (fetal bovine serum) and 1% penicillin-streptomycin at 37° C. and 5% CO2. For the generation of pseudo rho zero cells (0.0), U251 MG cells were treated with low dose (250 ng/ml) ethidiumbromide for 3 months. The cells were supplemented with DMEM (10% FCS, 1% P/S) containing 50 μg/ml L-pyruvate and 5 mg/ml uridine (King and Attardi, 1989).

### Animal Studies

All animal work was carried out in accordance with the NIH guidelines Guide for the Care and Use of Laboratory Animals. Six-week-old male athymic CD1 nude mice (Charles River, n=8) were injected subcutaneously with 7.5×106 U251MG cells in 100 μl PBS in the right flank using a 30-gauge needle. After 3 weeks, treatment of the xenograft tumors was initiated with 10 μg rhHMGB1 in 500 μl PBS or PBS only (control group) by daily intraperitoneal injections at the contralateral side for 6 weeks. Tumor volume was measured by a calliper using the ellipsoid formula (length×width×height×½) as described (Tomayko and Reynolds, 1989). After 6 weeks of treatment the animals were sacrificed and the paraffin embedded tumor sections were subjected to immunofluorescence analysis as outlined below.

### 1-D/2-D Electrophoresis

The U251 MG tumor cells were lysed in lysis buffer containing 20 mmol/L Tris-HCl (pH 7.4), 137 mmol/L NaCl, 10% (v/v) glycerine, 1% Triton X-100, 2 mmol/L EDTA, 100 mmol/L phenylmethylsulfonyl fluoride, and protease inhibitors (Complete mini; Roche Molecular Biochemicals). For the detection of phosphoprotein levels, 10 mmol/L NaPPi, 50 mmol/L NaF, and 20 mmol/L NaVO4 were added to the lysis buffer. After 15 min incubation on ice, the lysates were centrifuged at 14,000×g for 10 min. The total protein concentration of the lysates was measured using the Bradford assay (Bio-Rad). Soluble protein (25-100 μg per lane) was separated on 10% to 15% polyacrylamide gels and blotted onto nitrocellulose by standard procedures. The membranes were washed, incubated with primary antibody as indicated: mouse anti-human caspase-3 (1:500, Imgenex, San Diego, Calif.); polyclonal rabbit anti-human phosphorylated p44/42 mitogen-activated protein kinase and polyclonal rabbit anti-human phosphorylated stress-activated protein kinase/JNK (1:1,000, Cell Signaling Technology, Inc.); polyclonal anti-rabbit JNK 54/46 (1:1,000, Cell Signaling Technology, Inc.); polyclonal rabbit anti-human IkBalpha (1:500, Santa Cruz Biotechnology, Inc.); mouse monoclonal anti-human BNIP3 (1:10,000, R&D Systems, Inc.); mouse monoclonal anti-human phosphorylated-histone H2A.X (Upstate, Inc.); mouse monoclonal anti-human cytochrome c (1:100, BD Biosciences Pharmingen GmbH, Heidelberg, Germany); mouse anti-human cytochrome c oxidase complex IV (1:100, Molecular Probes, Invitrogen, Karlsruhe, Germany); polyclonal rabbit antimalondialdehyde (1:250, Calbiochem, Merck, Darmstadt, Germany) and mouse monoclonal antibodies to β-tubulin or β-actin (1:3,000, Sigma Chemical Co., St. Louis, Mo.), washed, and incubated with the appropriate secondary antibody (1:3,000; horseradish peroxidase-conjugated, Bio-Rad). Bound antibodies were visualized using an enhanced chemiluminescence detection system (GE-Healthcare, Uppsala, Sweden).

Mitochondrial extracts of U251 MG cells precipitated according to the ProteoExtract Protein Precipitation Kit (Calbiochem) were pipetted into the sample cup of each strip holder. For large format 2-D-electrophoresis with silver staining and 2-D-western blots, isoelectric focusing (IEF) was performed as described previously (Schutt et al., 2002) using dry polyacrylamide gel strips (IPG, 24 cm) with an immobilized pH gradient pH 3-10 and the IPGphor1 Isolectric Focusing System (Amersham Pharmacia Biotech). IPG strips were rehydrated in reswelling buffer (7 M urea, 2 M thiourea, 2% (w/v) CHAPS, 65 mM dithiothreitol (DTT), 0.8% Pharmalite) overnight. The IEF protocol started at 500 V for 4 h, followed by an increase from 500 V to 4000 V for 10 h and finally 8000 V for 10 h. Equilibration buffer for IPG strips contained 75 mM Tris-HCl, pH 8.8, 2% SDS, 6 M urea, 29.3% glycerol and 0.002% (w/v) bromophenol blue solution, supplemented with 1.5% DDT for the first 15 min and with 2.5% (w/v) iodoacetamide for the following 15 min. For the second dimension the ETTAN Dalt six electrophoresis system was used. After draining the equilibration solution on filter paper the IPG strips were placed on the SDS gel; the electrical settings were 10 mA/gel, 80 V, 1 W/gel for 1 h, followed by 40 mA/gel, 500 V, 13 W/gel for 5-6 h. For the 2-D-western blots the settings were 20 mA/gel, 600 V, 30 W/gel for 50 min, followed by increase of current up to 50 mA. After transfer the membranes were incubated with polyclonal rabbit anti-malondialdehyde antibody (1:250) overnight and bound antibodies were visualized using an enhanced chemiluminescence detection system (GEHealthcare). For silver staining the large format 2-D-gels were fixed for 1 h (absolute ethanol, glacial acetic acid), sensitized for 1 h (absolute ethanol, 1.25‰ (w/v) 4° C. glutaraldehyde, 0.2% (w/v) sodium thiosulfate, 6.8% (w/v) sodium acetate), washed with ddH2O for 5×8 min, stained for 1 h (0.25% (w/v) with silver nitrate solution, 0.15‰ (w/v) formaldehyde), washed with ddH2O for 4×1 min, developed for 23 min (2.5% (w/v) sodium carbonate, 0.15‰ formaldehyde), and stopped with 1.52% (w/v) Na2EDTA.H2O for 45 min.

### Cytotoxicity and Proliferation Assays

20,000 U251MG glioblastoma cells or human astrocytic cells (ScienCell) were plated into 96-well plates. After 24 h cells were treated with rhHMGB1 (Sigma Aldrich) or rat recombinant HMGB1 (generously provided by Angelika Bierhaus, Heidelberg, Germany), and TRAIL (for 24 h) or Temozolomide (for 72 h) alone or in combination, respectively. Cell survival was measured by crystal violet staining as described previously (Gdynia et al., 2007). Cell proliferation was assessed by seeding 50,000 U251 MG cells in 6 cm culture plates and treatment with rhHMGB1, followed by counting the cells in a Neubauer chamber at day 0, 2, 4 and 5.

### Southern and Dot Analysis

Subcellular fractionation was performed as described below. The DNA of the resulting mitochondrial suspension was purified by a modified method described in Almeida et al. (Almeida et al., 2006). Briefly, contaminating nuclear DNA was precipitated by lysis of the mitochondria in a buffer of 50 mM glucose, 1.5 mM EDTA, 25 mM Tris-HCl, pH 7.4 with 20% SDS and 3 M K-acetate. After centrifugation (10 min, 4° C., 10.000×g) the supernatant was digested with 20 mg/ml proteinase K for 1 h at 37° C. and mtDNA was extracted twice with phenol-chloroform and one additional time with chloroform, followed by precipitation of mtDNA from the aqueous phase by adding two volumes of isopropanol. The pellet was washed with 80% ethanol at −20° C. After drying the mtDNA was solved in TE-buffer (10 mM Tris-HCl, pH 7.4, 1 mM EDTA, pH 8.0). After treatment of the samples with 100 μg/ml ribonuclease A (RNAse A) for 1 h at 37° C., the phenol-chloroform extraction steps were repeated and mtDNA was precipitated by adding 2 volumes of 100% ethanol and 1/10 volume 5 M NaCl at −20° C. overnight. After a centrifugation step for 20 min at 4° C. with 13,000 rpm and washing the pellet 2× with 80% ethanol, followed by drying, the pellet was solved in ddH2O. The purified DNA was digested overnight with BamHI and loaded on a 0.5% agarose gel. The 16.5 kbp fragment visible under UV-light was excised from the gel using a scalpel and purified by using the Qiagen Gel Extraction Kit. This purified linearized mtDNA was random prime labelled with alkali-labile digoxigenin-dUTP (desoxyuridinetriphosphate) from the Dig High Prime Labeling and Detection Starter Kit II (catalogue number 11 585 614 910, Roche). Labeling efficiency was determined as described in the kit. Dot blot experiments were performed by pipetting the isolated mtDNA onto a positively charged nylon membrane (Roche) followed by hybridization with the DIG-mtDNA probes and detection of the labeled mtDNA with 1:10,000 Fab fragments from an antidigoxigenin antibody (Roche).

### Enucleation of Cells

10,000 U251MG cells were seeded in drops of DMEM (10% FCS, 1% P/S) on round cover slips and incubated for 24 h at 37° C. and 5% CO2. The cover slips were put upside down in a 15 ml Falcon tube containing growth medium complemented with 10 μg/ml cytochalasin B. After 1 h incubation at 37° C. the 15 ml tubes were centrifuged for 1.5-3 h at 37° C. with 4.000 rpm. Then, the cover slips were removed and put into 6 cm plates containing growth medium. After a recovering period of 8 h the cytoplasts were treated with rhHMGB1 for 24 h. The successful enucleation was confirmed by immunofluorescence with 1 μg/ml DAPI.

### FACS Analysis of Reactive Oxygen Species, Mitochondrial Membrane Potential, and Cell Cycle

For measurement of reactive oxygen species (ROS) and mitochondrial membrane potential (MMP) 1.5×106 U251 MG cells were seeded in 6 cm plates, treated with 40 nM rhHMGB1 for 24 h and incubated with the fluorescent DiOC6 (3,3′-dihexyloxacarbocyanine iodide, 40 nmol/ll, Sigma Chemical Co., St. Louis, Mo.) or with the fluorescent H2DCF-DA (dichlorodihydrofluorescein diacetate, 10 μM, Invitrogen) for 1 h at 37° C., respectively. The cells were trypsinized and centrifuged at 2,000 rpm for 5 min at 4° C. The supernatant was discarded and the pellet was washed 2× with sterile Dulbecco's PBS containing 0.1% fetal calf serum on ice and again centrifuged at 2,000 rpm for 5 min at 4° C. The pellet was resuspended in 500 μl sterile Dulbecco's PBS on ice. Oxidation of H2DCF to DCF by ROS and DiOC6 accumulation in mitochondria were measured immediately at FL-1 by flow cytometry using a FACScalibur (Beckton Dickinson, Heidelberg, Germany). Cell cycle was determined by measurement of priopidiumiodide (PI)-uptake of the cells according to standard procedures. Briefly, the cells were harvested and washed twice in sterile Dulbecco's PBS on ice, spun down at 2,000 rpm for 5 min at 4° C. and fixed with 70% ethanol for 30 min at 4° C. Contaminating RNA was eliminated by treatment with 100 μg/ml of RNase A, then 50 μg/ml PI was added and DNA content was measured immediately at FI-2. The G2 arrest positive control was generated by treating 1.5×106 U251MG cells in a 6 cm plate with nocodazole (100 ng/ml) for 24 h.

### Plasmid Transfection

U251MG cells were seeded in 96-well plates (15,000 per well) and rapid lipofectamine transfection was performed as described earlier (Gdynia et al., 2008) with plasmids (pcDNA3 vector) encoding for human RAGE (receptor of advanced glycolisation endproducts), .RAGE (lacking the cytoplasmatic domain), YFP-tagged TLR2 and YFP-tagged TLR4 (Toll-like receptor 2 and 4), myc-tagged MyD88 (Myeloid differentiation primary response gene 88) and myc-tagged TRIF (Toll/IL-1 receptor domain-containing adaptor inducing IFN-beta). The controls were transfected with the pcDNA3 neo plasmid. After transfection cells were treated with rhHMGB1 for 72 h or left untreated and cell survival was measured by the crystal violet method (Gdynia et al., 2007). In some experiments, cells were treated with the physiological agonists of the indicated receptors for 72 h: 1 μg/ml LPS (lipopolysaccharide), 1 μg/ml PAM3CSK4 (N-palmitoyl-S-[2,3-bis(palmitoyloxy)-(2R,S)-propyl]-(R)-cysteinyl-seryl-(lysyl) (3)-lysine) or 100 μg/ml AGE (advanced glycolysation end product).

### Iodine-125 Isotope Labelling

U251 cells were seeded on 10 cm culture dishes. After treatment of cells with 80 nM of iodine-125 coupled rhHMGB1 for 0 h, 4 h, 16 h and 24 h, the cells were detached carefully by incubation with cell dissociation buffer (30 min) in order to protect possible surface proteinreceptor-complexes, harvested, and subjected to subcellular fractionation using the ApoAlert Fractionation Kit as described below (the plasma membrane fraction was extracted from the cytoplasmatic fraction by a last centrifugation step of 400.000×g for 15 min at 4° C.). Successful fractionation was confirmed by western blot. Successful coupling of the HMGB1-tyrosine residues with I-125 was shown by the high radioactivity in the protein fraction of sephadex G25 gel filtration (early eluates). Fraction 4 was used for the incubation of cells with I-125-HMGB1. For radioactivity measurements cells were solubilized in 0.5M NaOH and mixed with 10 ml LSC cocktail (Ultima Gold, Perkin Elmer, Waltham Mass.). The radioactivity of each subcellular fraction was measured in a liquid scintillation counter (Packard TriCarb 2900, Meriden, Ill.) by using the Compton effect.

### Cellular Fractionation

Enrichment of mitochondrial fractions was performed using the ApoAlert Cell Fractionation Kit (Clontech, Mountain View, Calif.) as described previously (Gdynia et al., 2008). Briefly, after centrifugation and several washing steps, the cell pellet was resuspended in ice-cold 1× Fractionation Buffer Mix containing protease inhibitors (Complete mini; Roche Molecular Biochemicals, Indianapolis, Ind.) and 1 mM 1,4-Dithio-DL-threitol solution (DTT). After 10 min incubation on ice, cells were homogenized with a dounce tissue grinder. Efficiency of homogenization was controlled by microscopy. The homogenate was centrifuged at 700×g for 10 min at 4° C. and the resulting supernatant at 10,000×g for 25 min at 4° C. The pellet containing the mitochondrial fraction was resuspended in 1× Fractionation Buffer Mix.

### Immunofluorescence and Confocal Microscopy

U251MG cells were plated onto 8 well chamber-slides (Nunc, Wiesbaden, Germany) or grown confluently in 6 cm culture dishes containing round glass cover plates. For some samples, the cells were stained with the fluorescent dye MitoTracker Red CMXRos (50 nM, Molecular Probes, Invitrogen, Karlsruhe, Germany) for 30 min at 37° C. 24 h after seeding, the cells were fixed for 15 min with 2% paraformaldehyde solution or pre-cooled 100% methanol (on ice), washed 3× with PBS, permeabilized on ice with PBS containing 0.2% TritonX-100 and 1% fetal calf serum for 5 min and washed again three times with PBS containing 1% or 5% FCS. After blocking with PBS containing 1% or 5% FCS at room temperature for 60 min, permeabilized cells were incubated with monoclonal rabbit fluoro-conjugated anti-COX IV antibody (1:10, 3E11, Alexa Fluor 488 conjugate, Cell Signaling) or monoclonal mouse anti-polyhis-tag antibody (1:1000, R&D Systems) for 30 min at 4° C. and then washed three times with PBS with 1% or 5% FCS. In case the primary antibody was unconjugated to a fluorescent dye, goat anti-mouse or anti-rabbit secondary antibody Alexa Fluor® 488 or 564 (1:200, Invitrogen GmbH, Karlsruhe, Germany) together with 1 μg/ml 4′,6-Diamidino-2-phenylindol (DAPI) was added for 30 min at 4° C. in a dark chamber. After washing three times with PBS with 1% or 5% FCS, cells were covered with cover slips. Photomicrographs were taken using a Olympus VANOX-T (AH-2) fluorescence microscope with immersion oil, ne 23=1.518 and the Soft Imaging System AnalysisB software (Leica Microsystems, Wetzlar, Germany) or on a confocal microscope (Leica SP2).

### Immunohistochemistry

Paraffin embedded slices of mouse tissue or xenograft tumors were dewaxed and dehydrated using xylene and ethanol, followed by heat-induced antigen retrieval using 10 mM citrate buffer, pH 6.0, for 3×5 min in a cooker. The sections were incubated with 1:10 COX IV (Alexa Fluor® 488 conjugate) antibody over night at 4° C., washed with PBS and covered with Vectashield® mounting medium (H-1200, Vector Laboratories Inc., 94010 CA, USA) containing DAPI (1.5 μg/ml).

### Electron Microscopy

Cells were fixed in situ with 2.3% glutaraldehyde in 50 mM sodium cacodylat (pH 7.2) for 30 min at 4° C., scraped from the plates, collected by low-speed centrifugation at 200×g for 10 min at 4° C. and successively stained with 2% osmium tetroxide and 0.5% uranyl acetate. After dehydration and embedding in Epru the probes were processed for ultrathin section. Micrographs were taken with a Zeiss EM-10A electron microscope at 80 kV. The magnification indicator was routinely controlled by the use of a grating replica.

### Statistical Analysis

All data are mean±SD. The significance of differences between groups was determined by Student's t test. P values <0.05 were considered statistically significant.

### Example 2

### Recombinant Human HMGB1 Exerts Cytotoxic Effects on Cancer Cells

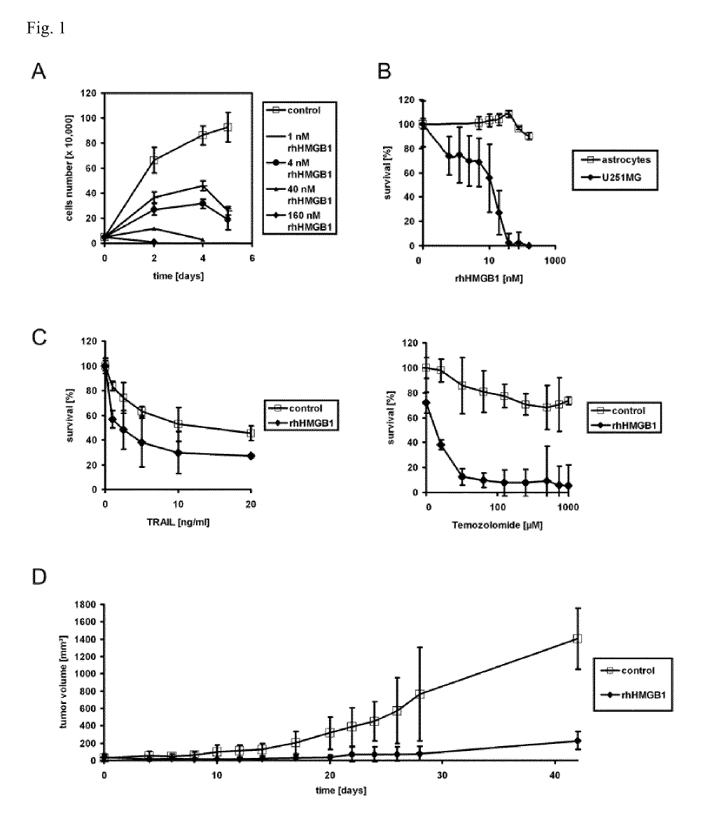

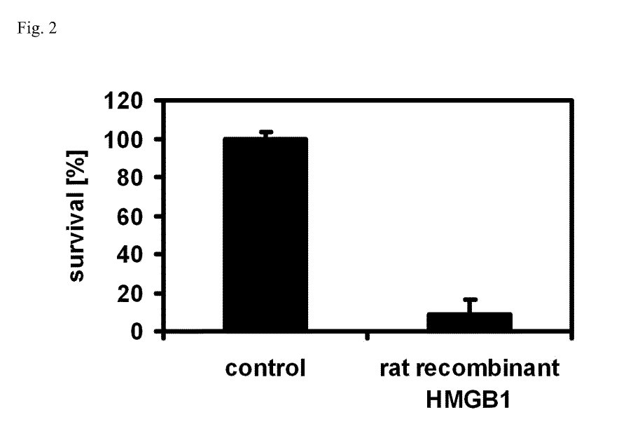

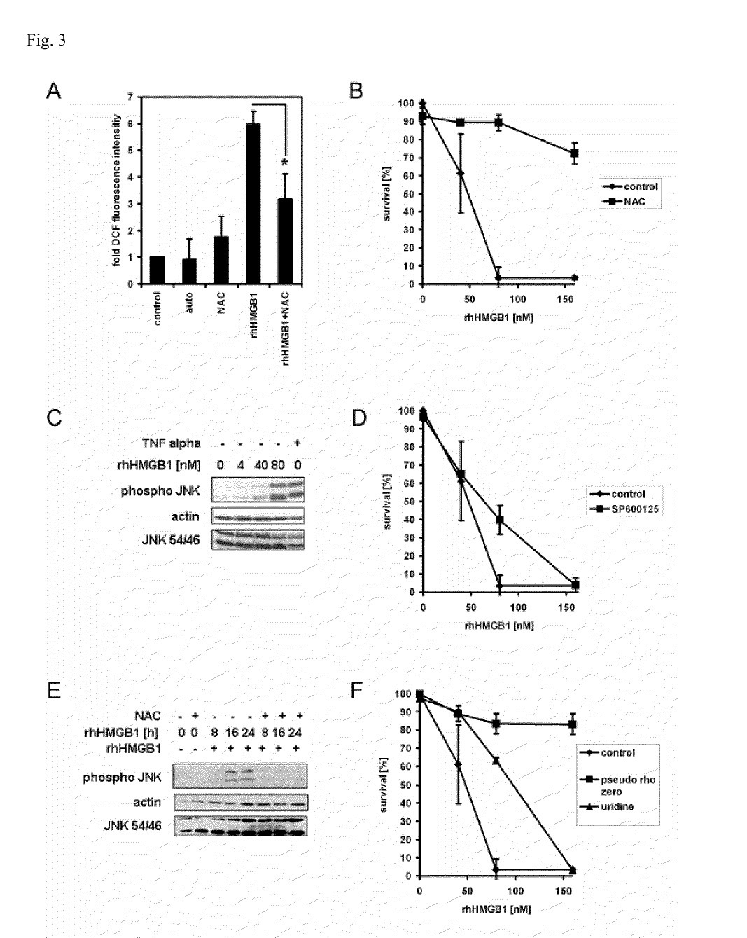

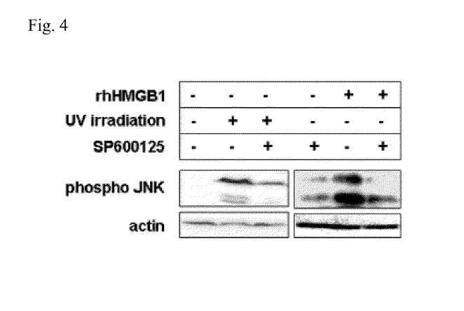

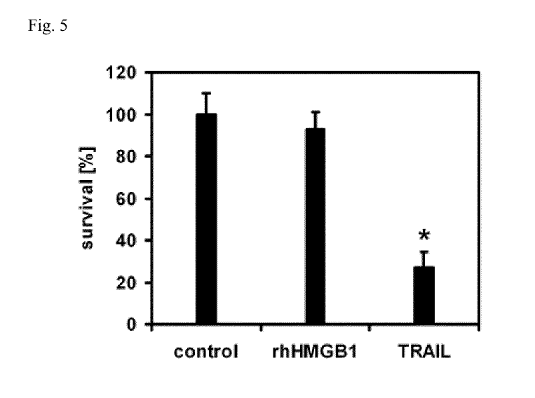

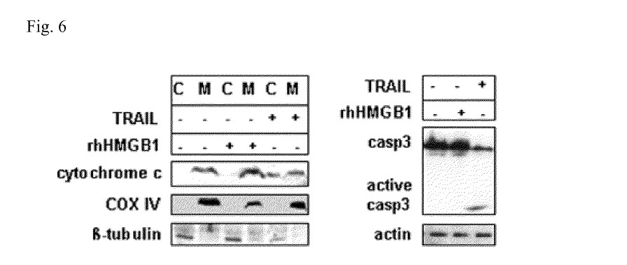

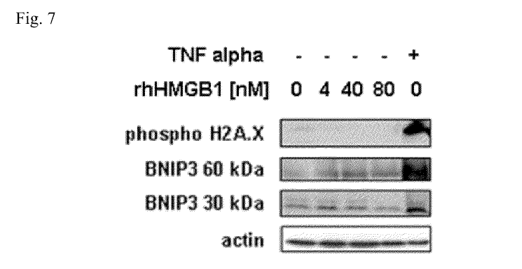

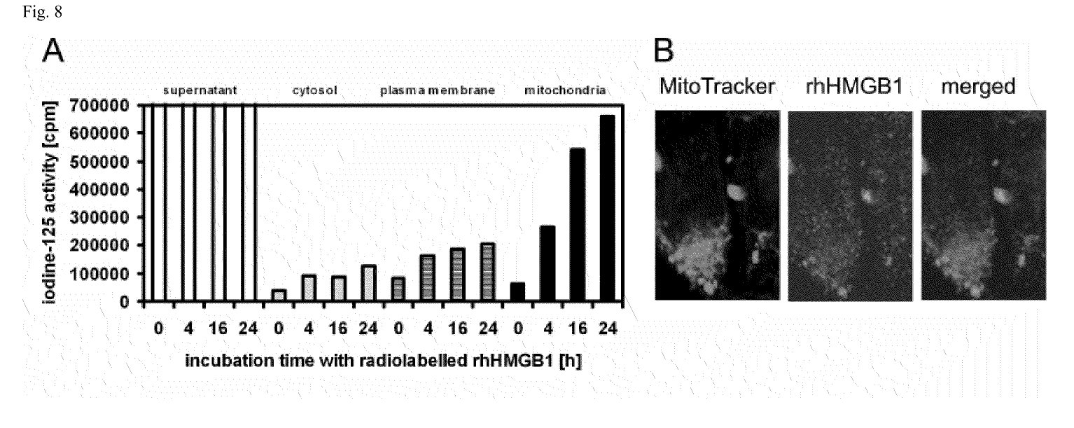

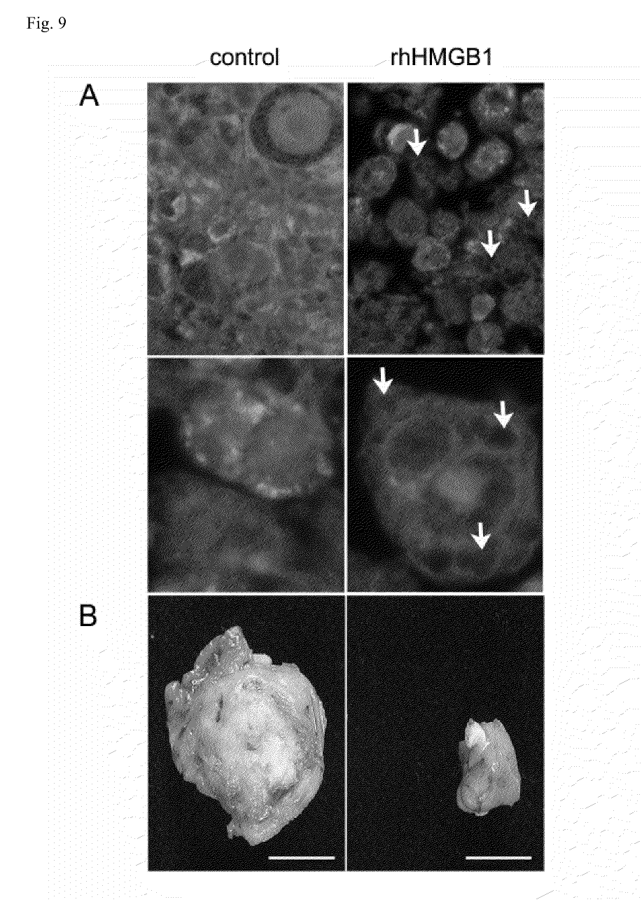

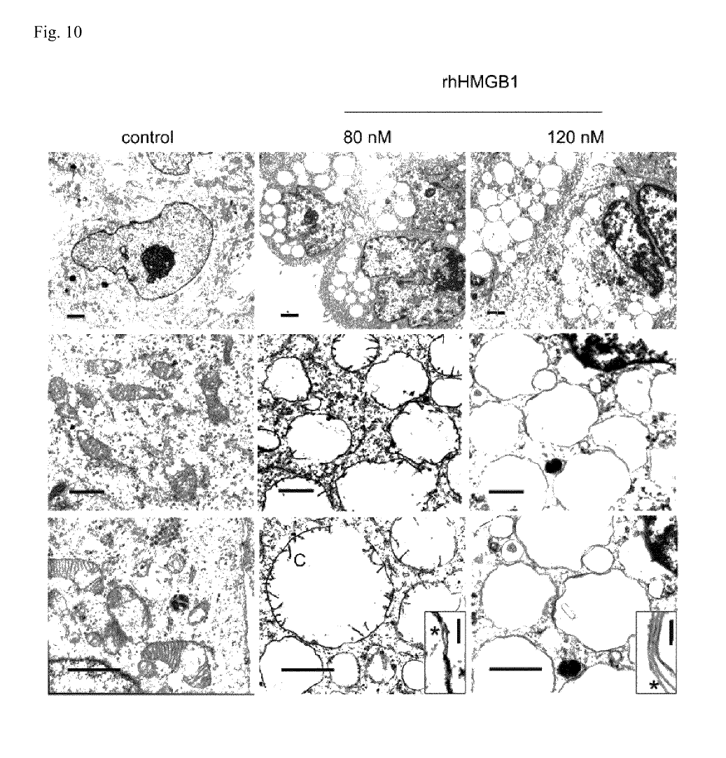

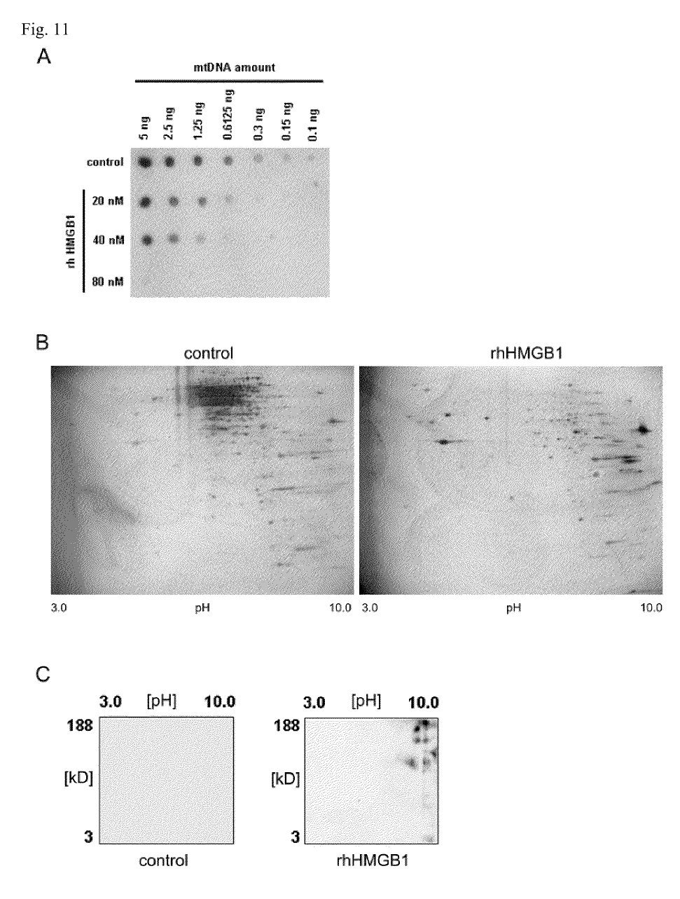

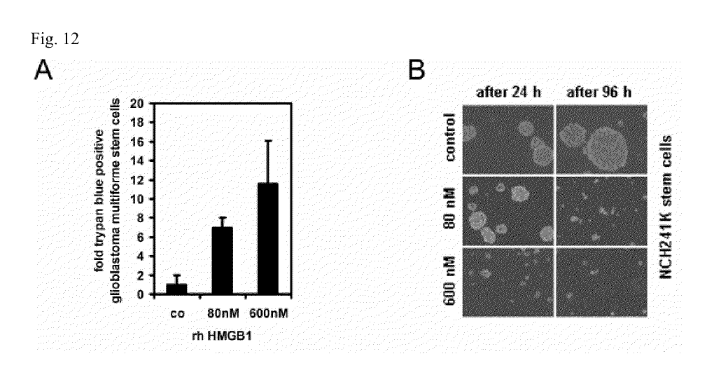

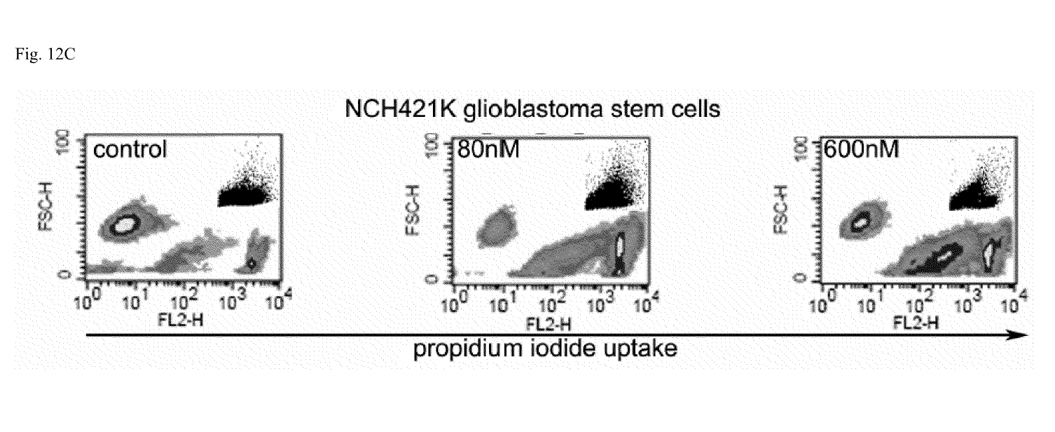

To study the cellular effects of exogenous rhHMGB1 human glioblastoma cells were treated with increasing concentrations of rhHMGB1 for several days (FIG. 1A). Treatment with rhHMGB1 resulted in a strong inhibition of tumor cell proliferation and induction of cell death even at low nanomolar concentrations. Cytotoxicity assays revealed that U251 MG glioblastoma cells, but not human non-immortalized astrocytes were susceptible to rhHMGB1-induced cell death (FIG. 1B). Further, combined treatment of glioma cells with rhHMGB1 and the death ligand TRAIL or the anti-cancer drug Temozolomide resulted in a strong synergistic induction of cell death (FIG. 1C). Rat recombinant HMGB1 protein which exhibits a high homology to human HMGB1 (>98%) showed similar cytotoxic effects as rhHMGB1 (FIG. 2). Next, the anti-cancer activity of rhHMGB1 was tested in vivo. To this end, CD1 athymic nude mice bearing subcutaneous glioma xenograft tumors were treated systemically with rhHMGB1. After six weeks, the rhHMGB1-treated animals showed significantly smaller tumors than the control group (FIG. 1D).

Furthermore, it was shown in a trypan blue vital stain that NCH421 K glioblastoma stem-cell-like cells treated with 80 nM or 600 nM rhHMGB1 for 24 h had significantly higher trypan blue influx than the untreated control cells (FIG. 12A, p<0.05, 80 nM=7±1 fold increase and 160 nM=11.56±4.51 fold increase versus control respectively), demonstrating the beginning break-down of plasma membrane integrity. Trypan blue influx into NCH421 K glioblastoma multiforme stem-cell-like cells was measured by counting blue cells in a Neubauer chamber 24 h after treatment with 80 nM and 600 nM rhHMGB1 of 500,000 cells in mini culture flasks and then incubating them for 10 min with 1:1,000 trypan blue solution. Propidiumiodide (PI) uptake in NCH421 K cells treated with 80 nM or 600 nM rhHMGB1 was evidently increased compared to the untreated control using FACS analysis (FIG. 12C). The strong anti-proliferative effect resulted in clearly smaller and fewer spheroid cell aggregates (FIG. 12B). Glioblastoma stem-cell-like cell line NCH421K was maintained in BIT (bovine serum albumine, insulin, transferring)-medium (DMEM HAMS F12+1% P/S+1% L-glutamin+10 ml BIT+EGF (20 ng/ml)+BFGF (20 ng/ml)).

### Example 3

### RhHMGB1-Dependent Increase in Radical Oxygen Species Leads to JNK Activation

Next, the intracellular signalling events leading to rhHMGB1-induced cell death were examined. RhHMGB1 treatment resulted in a strong generation of radical oxygen species (ROS) which was partially prevented by the ROS scavenger NAC (FIG. 3A). Co-treatment of the cells with NAC substantially inhibited rhHMGB1-induced cell death (FIG. 3B). Moreover, rhHMGB1 caused a continuous and dose dependent phosphorylation of c-Jun N-terminal kinase 1/2 (JNK) (FIG. 3C). The activation of JNK contributes to rhHMGB1-dependent cytotoxicity since co-incubation with the specific JNK inhibitor SP600125 not only reduced the amount of phosphorylated JNK (FIG. 4) but also partly inhibited cell death induced by rhHMGB1 (FIG. 3D). The NAC-dependent inhibition of ROS prevented the activation of JNK after rhHMGB1 treatment (FIG. 3E), indicating that rhHMGB1-induced ROS generation occurs upstream of JNK activation. To investigate the functional importance of intact mitochondria in rhHMGB1-induced cytotoxicity, cells devoid of intact mitochondrial DNA, so-called pseudo rho zero cells (0.0 cells, see Example 1), were generated. Importantly, 0.0 cells were almost completely resistant to rhHMGB1-induced cell death (FIG. 2F), whereas other cell death mechanisms, such as apoptotic signalling upon treatment with the death ligand TRAIL was still intact in these cells (FIG. 5). These results suggest that the mitochondria represent a main target for cytotoxic actions of rhHMGB1. This hypothesis was further confirmed by the observation that rhHMGB1-induced cell death in U251 MG glioma cells was substantially inhibited by adding uridine to the culture medium (FIG. 3F). Whereas 0.0 cells devoid of intact mitochondrial DNA are pyrimidine auxotrophs, normal cells essentially depend on mitochondrial-derived uridine. Therefore, the uridine mediated protection from rhHMGB1-induced cell death indicates that the disturbance of mitochondrial metabolic pathways play an important role in the cytotoxic actions of rhHMGB1.

To further dissect the mechanisms of rhHMGB1-induced cytotoxicity, it was examined whether rhHMGB1-induced cell death shares the characteristics of apoptosis, necrosis, or senescence. Firstly, not detect the typical morphological features of apoptosis after rhHMGB1 treatment of cells could not be detected, such as membrane blebbing, formation of apoptotic bodies, or chromatin condensation. Secondly, neither activation of caspase 3 nor release of cytochrome c was observed after rhHMGB1 treatment (FIG. 6). Next, it was examined whether the senescence-associated DNA double strand break repair enzyme H2A.X is activated by rhHMGB1. However, H2A.X phosphorylation was not observed upon rhHMGB1 treatment (FIG. 7). Similarly, the necrosis-associated BCL2/adenovirus E1B 19 kDa interacting protein 3 (BNIP3) was not up-regulated after treatment of cells with rhHMGB1 (FIG. 7).

### Example 4

### RhHMGB1 Localizes to the Mitochondria and Induces the Formation of Megamitochondria

Given the mitochondria-dependent effects of rhHMGB1, the subcellular translocation events after treatment of cells with rhHMGB1 was investigated. To this end, U251MG glioma cells were incubated with iodine 125-labelled rhHMGB1 followed by subcellular fractionation. This experiment showed a time dependent increase of radioactivity in the mitochondrial fraction (FIG. 8A), suggesting that after penetration of the cell membrane a substantial amount of rhHMGB1 translocates to the mitochondria. Mitochondrial localization of rhHMGB1 was also confirmed by immunofluorescence microscopy after co-staining with a mitochondrial marker (FIG. 8B). Since treatment of glioma cells with rhHMGB1 resulted in the formation of large vacuole-like organelles as observed by phase contrast microscopy, immunofluorescence studies with the mitochondrial marker enzyme COX IV were performed. These experiments revealed that rhHMGB1 treatment resulted in the generation of extremely enlarged spherical mitochondria (FIG. 8C). Importantly, the formation of megamitochondria was also observed in the tumor tissue of xenografted nude mice treated systemically with rhHMGB1 (FIG. 9).

To confirm the rhHMGB1-dependent formation of megamitochondria, electron microscopy of cells treated with rhHMGB1 was performed. The ultrastructure of the vacuoles revealed a double membrane and rudimentary cristae (FIG. 10) confirming the nature of mitochondria. The number and size of megamitochondria increased in a time- and concentration-dependent manner after incubation with rhHMGB1. In contrast, autophagosomes characteristic for autophagic cell death or disintegration and rupture of the outer cell membrane indicative of necrotic cell death were not observed.

### Example 5

### RhHMGB1 Rapidly Depletes Mitochondrial DNA and Severely Damages the Mitochondrial Proteome

To further investigate the effects of rhHMGB1 on mitochondria, the mitochondrial DNA content of cells after rhHMGB1 treatment was examined. Dot blot analysis demonstrated a rhHMGB1-dependent substantial and rapid depletion of mtDNA (FIG. 11A). Digestion of the mtDNA from the untreated sample by the human mtDNA single cutter restriction enzyme BamHI resulted in a strong band at 16.5 kbp (linear mtDNA) in a digoxigenin southern blot, confirming the mitochondrial origin of the 23 kbp band (data not shown). The rapid mtDNA depletion after rhHMGB1 treatment was accompanied by a massive depletion of the isoelectric-neutral proteins and an increase of predominantly alkaline protein spots in a 2-D-silver staining of the mitochondrial proteome (FIG. 11B). Moreover, 2-D-western blot analysis of the mitochondrial proteome revealed an substantial increase in covalently bound malondialdehyde in the alkaline range after treatment with rhHMGB1 (FIG. 11C).

1. A method for treating a subject suffering from cancer comprising administering to said subject a therapeutically effective dose of High Mobility Group B1 (HMGB1) protein.

2. The method of claim 1, further comprising administering a therapeutically effective dose of at least one chemotherapeutic agent and/or at least one biotherapeutic agent and/or at least one virotherapeutic agent.

3. The method of claim 1, further comprising surgical treatment and/or radiotherapy.

4. The method of claim 1, wherein the cancer is a glioblastoma or a colon carcinoma.

5. A method for treating a subject suffering from cancer comprising administering to said subject a therapeutically effective dose of a polynucleotide comprising a coding region for an HMGB1 gene in an expressible form.

6. The method of claim 5, further comprising administering a therapeutically effective dose of at least one chemotherapeutic agent and/or at least one biotherapeutic agent and/or at least one virotherapeutic agent.

7. The method of claim 5, further comprising surgical treatment and/or radiotherapy.

8. The method of claim 5, wherein the cancer is a glioblastoma or a colon carcinoma.

9. The method of claim 5, wherein said polynucleotide is comprised by a vector.

10. The method of claim 5, wherein said polynucleotide is comprised by a host cell which is administered to the subject.
